(19) United States
(12) Patent Application Publication
(10) Pub. No.: US 2023/0203593 A1
(43) Pub. Date: Jun. 29, 2023

(54) METHOD FOR CARRYING OUT IN VITRO MOLECULAR DIAGNOSIS OF OVARIAN TUMOR AND KIT

(71) Applicants: UNIVERSITÀ DEGLI STUDI DI BARI (50%), Bari BA (IT); ITEL TELECOMUNICAZIONI S.R.L. (50%), Ruvo di Puglia (BA) (IT)

(72) Inventors: Antonio SCILIMATI, Bari BA (IT); Maria Grazia PERRONE, Bari BA (IT); Savina FERORELLI, Bari BA (IT); Anna Maria DE GRASSI, Bari BA (IT); Giuseppe PERRONE, Roma (RM) (IT); Francesca ZALFA, Roma (RM) (IT); Michele DIAFERIA, Ruvo di Puglia (BA) (IT); Vincenzo DIMICCOLI, Ruvo di Puglia (BA) (IT)

(21) Appl. No.: 17/925,995

(22) PCT Filed: May 19, 2021
(86) PCT No.: PCT/IB2021/054315

§ 371 Date: Nov. 17, 2022

(30) Foreign Application Priority Data
May 21, 2020 (IT) 102020000011977

(51) Int. Cl. C12Q 1/6886

(52) U.S. Cl. CPC C12Q 1/6886; C12Q 2600/158; C12Q 2600/118; C12Q 1/6837

(57) ABSTRACT

The present invention relates to a method for performing an in vitro molecular diagnosis of ovarian cancer in a subject. Furthermore, the present invention relates to a kit allowing to perform an in vitro diagnosis of ovarian cancer.

### ABSTRACT

The present invention relates to a method for performing an in vitro molecular diagnosis of ovarian cancer in a subject. Furthermore, the present invention relates to a kit allowing to perform an in vitro diagnosis of ovarian cancer.

### STATE OF THE ART

According to the latest data from the American Cancer Society and AIOM (Italian Association of Medical Oncology), ovarian carcinoma is the seventh most frequent neoplasm in the female population and the leading cause of death from gynecological cancer in industrialized Countries.

The American Cancer Society estimates that approximately 21,750 new cases of ovarian carcinoma will be diagnosed in 2020 and 13,940 women will die of ovarian carcinoma in the United States. In fact, in the United States, ovarian carcinoma is the second most common gynecologic cancer (affecting about 1/70 women).

The ovary is the female gonad consisting of an even, symmetrical gland located alongside the uterus.

Each of the two ovaries has an upper (or tubal) pole joined to the infundibulum of the uterine tube, and a lower (or uterine) pole joined to the uterus by the uterine-ovarian ligament.

There two layers of tissue that constitute the ovaries: the inner layer (medullary part) rich in dense connective tissue and blood vessels for the irrigation and nourishment of the ovary itself and the outer layer of tissue (cortical part) containing the ovarian follicles involved in the production and maturation of the oocyte, and the subsequent expulsion of an ovum.

The ovarian cancer is due to the uncontrolled proliferation of the cells of the ovarian organ: epithelial cells, stromal cells and germ cells.

There are three types of malignant ovarian cancer, which are related, precisely, to the type of cells that constitute the ovary. The epithelial tumors originating from the epithelial cells of the ovary account for about 85-90% of the so-called ovarian tumors, whereas the germ cell tumors, which develop from the cells involved in the production of oocytes, are rarer and may more frequently affect adolescents and young women. The tumors of the stroma and sex cord represent the third type of malignant ovarian cancer and are rare tumors, originating from the supporting tissue of the ovary, involved in the production of estrogens and progesterone.

The ovarian cancer is often fatal as it is asymptomatic. Therefore, at the time diagnosis the tumor is often already in an advanced stage.

The diagnosis is based on the histological examination of the biopsy specimen taken from the ovary or surrounding areas, and its subsequent staging. By diagnostic imaging (CAT, magnetic resonance or PET) it is then possible to precisely determine the extent of the tumor. Waiting for the results of the histological examination and diagnostic imaging may delay the diagnosis. In fact, often several weeks pass before having a diagnosis of ovarian cancer which, as already mentioned, is frequently already in an advanced stage.

Therefore, there is a need to have a method that allows quick and accurate diagnosis of ovarian cancer.

The treatment of ovarian cancer requires hysterectomy (removal of the uterus), bilateral salpingo-oophorectomy, excision of the tumoral tissue and, when necessary, chemotherapy. Especially after hysterectomy, it is necessary to evaluate the presence of further tumor cells that could resume proliferation and give recurrence. However, there are currently no commercially available kits for testing the efficacy of the surgical or pharmacological treatment of the disease.

Even before kits for testing the efficacy of the surgical or pharmacological treatment of the disease are available, there are no kits that can assess, in the shortest possible time, an exact diagnosis of ovarian cancer.

Given the increasing incidence of ovarian cancer in the industrialized countries, the lack of symptoms related to its onset and the need to treat the tumor at an early stage (in order to identify its stage and decide, consequently, the type of treatment to be used), sensitive, accurate diagnosis methods with short waiting times are required. There is also a need for methods to quickly enable to define the prognosis of a patient with ovarian cancer in order to assess the type of therapeutic treatment best suited to the aggressiveness of the tumor of said patient.

### OBJECTS OF THE INVENTION

An object of the present invention is to provide a method which enables making an in vitro diagnosis of ovarian cancer in a subject in a short time.

Another object of the present invention is to provide a method which enables identifying the prognosis of a subject suffering from ovarian cancer.

A further object of the present invention is to provide a kit which enables making an in vitro diagnosis of ovarian cancer in a subject. Still an object of the present invention is to provide a kit which enables defining the prognosis for a subject suffering from ovarian cancer.

### DESCRIPTION OF THE INVENTION

The objects above, as well as other objects, are achieved by means of the subject matter of the present invention, namely a method for making an in vitro diagnosis of ovarian cancer in a subject by determining, within one or more biological samples which have been collected and isolated, the expression levels of a pattern of genes identified as being differentially significantly expressed in ovarian cancer samples compared to healthy samples.

Thus, an object of the present invention is a method for performing an in vitro diagnosis of ovarian cancer, comprising the steps of:

a) determining, in one or more isolated samples, the expression levels of at least one gene and/or of at least one protein encoded by said at least one gene;

b) determining the expression levels of the same genes and/or of the same proteins in one or more reference samples;

c) comparing the expression levels determined in step a), with the expression levels determined in step b),

characterized in that said at least one gene is selected from ATP6V1B1, KRT18, KRT8, DSP, HMGA1, CLDN7, CLDN3, CLDN4, ELF3, ESRP1, MAL2, ST14, EPCAM, MYH14, FAM83H, PRSS8, SPINT1, CELSR2, SORT1, GPR56/ADGRG1, WFDC2, KLK6, KLK7, CP, MUC16, SLC34A2, UCP2, SCNN1A, SOX17, PAX8, S100A1, ITGB4, KRT7, FOLR1, MSLN, CDH1, TACSTD2, PTGS1, MIF, THSD4.

According to the method of the invention, said genes enable detecting with extreme precision the possible presence of ovarian cancer in one or more isolated biological samples, said one or more isolated samples being samples of ovarian tissue, for example biopsies of ovarian tissue.

In an alternative embodiment, the method of the invention is performed on isolated blood (liquid biopsy) or urine samples. The method of the invention, when carried out on blood or urine belonging to the subject undergoing the diagnosis, enables for certain diagnostic results to be obtained in a short time. Furthermore, another advantage of the liquid or urine biopsy is the ability to perform molecular analysis when, for some reason, isolated biological tissue is not available, e.g. biopsy of ovarian tissue.

The analysis of the liquid biopsy from urine and/or blood samples can be advantageously used for the identification of prognostic, or predictive, markers, and therefore the method of the invention, even when carried out on blood or urine belonging to the subject undergoing diagnosis/prognosis, also enables prognostic results to be obtained in a short time. In fact, the method of the invention performed on an isolated blood or urine sample advantageously enables the measurement of gene expression levels on possible circulating tumoral RNA, said circulating tumoral RNA being released into the blood by the tumor cells undergoing cell death, for example by apoptosis, or through other release mechanisms of cellular material, for example exosomes.

Therefore, the measurement of the expression level of genes according to the method of the invention can advantageously be carried out on RNA extracted from the isolated sample (e.g. blood or urine). In a particularly preferred embodiment, said RNA extracted from the isolated sample is analyzed by using nCounter Nanostring Analysis System technology, which allows direct multiplex measurements of gene expression characterized by high levels of precision and sensitivity.

According to an alternative embodiment, RNA extracted from the isolated sample is back-transcribed into cDNA according to known methods and analyzed by technologies other than nCounter Nanostring Analysis System. According to said alternative embodiment, cDNA obtained from the RNA extracted from an isolated sample (possibly tumoral RNA) can be quantified, for example, by using RNA-seq.

The genes enabling the discrimination of a tumoral ovarian tissue from a normal ovarian tissue according to the method of the invention have been identified thanks to studies on public platforms of expression profiles. Said public platforms of expression profiles contain the average value of the expression level of thousands of genes, detected in different cell types, said cell types being cancer cells or healthy cells. Furthermore, in particular, said platforms contain the average expression value of thousands of genes detected in different tumor types.

In the present invention, the term “normal ovarian tissue” means tissue not affected by tumor condition. In other words, the normal ovarian tissue is a healthy ovarian tissue. Thus, the healthy ovarian tissue is a tissue unaffected by condition, particularly unaffected by tumor condition, that exhibits physiological gene expression. Therefore, the cells in a normal ovarian tissue have physiological levels of gene expression, both of constitutive and specialized genes. The constitutive genes (also called housekeeping genes) are genes that are actively transcribed and translated at high level, as they encode for proteins and enzymes that are essential for the life of the cell, whereas the specialized genes are tissue-specific genes expressed only in that precise cell type or at a particular time in the cell life (e.g. during the differentiation). The normal ovarian tissue, in contrast to tumoral ovarian tissue, has physiological expression levels of both constitutive and tissue-specific genes.

In particular, the genes having diagnostic value according to the method of the invention have been identified thanks to a preliminary analysis (meta-analysis) of gene expression data on public platforms of gene expression profiles and a validation on isolated samples by using the method of the invention.

For the preliminary analysis, the expression profile of 18,734 genes in tumoral ovarian tissue and in normal (healthy) ovarian tissue was derived from large scale public platforms of gene expression profiles: the expression levels of genes in tumoral ovarian tissues have been taken from the TGCA (The Cancer Genome Atlas) platform, and the expression levels of genes in healthy ovarian tissues have been taken from the GTEx (Genotype-Tissue Expression) platform.

Said platforms show the expression levels, obtained by RNA-seq, of 18,734 genes in 266 tumoral ovarian tissues and 97 normal ovarian tissues.

At this point, we proceeded by identifying genes whose average expression level was simultaneously present in 5% of the most expressed genes in ovarian cancers and 25% of the least expressed genes in normal ovarian tissues. From this selection, 41 genes having diagnostic value according to the method of the invention were identified.

The diagnostic capability of said 41 genes has been validated by using the method of the invention. In other words, the expression level of said 41 genes was evaluated in 42 samples of tumoral ovarian tissue and compared with the expression values of the same genes in 3 normal, i.e., healthy, ovarian tissues (Example 1).

According to an embodiment of the method of the invention, step a) of detecting the expression levels of at least one of the genes according to the method of the invention is implemented by quantifying RNA extracted from an isolated sample. Therefore, the method of the invention comprises the step a′) of extracting nucleic acid from one or more isolated samples, upstream of step a). When said isolated sample is a biopsy sample of ovarian tissue, the nucleic acid extracted from said biopsy sample may be the total RNA of the sample.

When the isolated sample is blood, the nucleic acid extracted from said sample may be circulating RNA, possibly tumoral RNA, if present. Said circulating tumoral RNA possibly present is analyzed to assess the expression level of at least one of the genes having diagnostic value according to the method of the invention.

The quantification of the expression level of the nucleic acid extracted from one or more isolated samples according to the method of the invention, can be carried out by using in vitro methods, for example, conventional methods exploiting the hybridization of RNAs extracted from a sample, previously collected and isolated, obtained from said subject. Said quantification of the expression level of the nucleic acid extracted from one or more isolated samples can be performed by nCounter Analysis System technology by Nanostring, or by microarray, RNA-seq or other analysis platforms of the gene expression level, such as for example high throughput screening (or next generation sequencing) sequencers that provide for the quantification of RNA as such or back-transcribed into cDNA. RNA as such can be quantified as such by nCounter Analysis System technology by Nanostring. RNA can be back-transcribed into cDNA when its expression level is quantified e.g. by using RNA-seq.

In a particularly preferred embodiment, the detection of expression levels of at east one of the genes according to the method of the invention, implemented by quantifying RNA extracted from isolated sample, is carried out by a technology using molecular barcodes to quantitatively determine hundreds of transcripts in a single reaction, said technology being the Nanostring nCounter Analysis System technology. In fact, said system allows direct multiplex measurements of gene expression, which are characterized by high levels of precision and sensitivity (<1 copy/cell).

The Nanostring nCounter Analysis System associates a different barcode (having a particular color combination) to each transcript extracted from the isolated sample, said barcode being an identification code of the transcript under analysis.

In particular, the Nanostring nCounter technology uses molecular barcodes to quantitatively determine hundreds of transcripts in a single reaction, said technology being the Nanostring nCounter Analysis System technology. In fact, said system allows direct multiplex measurements of gene expression, which are characterized by high levels of precision and sensitivity (<1 copy/cell).

The Nanostring nCounter Analysis System associates a different fluorescent “barcode” (having a particular color combination) with each transcript extracted from the sample under examination, said barcode being an identification code of each particular transcript under analysis. The Nanostring nCounter Analysis System uses specific gene probe pairs that hybridize to mRNAs in the sample. Said two probes, reporter probe (approximately 50 nucleotides) and anchor (capture) probe (approximately 50 nucleotides), are DNA probes that hybridize to a region of approximately 100 base pairs of each specific RNA present in the sample. The reporter probe carries the fluorescent “barcode”, whereas the anchor probe ends with a biotin molecule and enables the probe+mRNA complex to be immobilized on a streptavidin-coated cartridge in order to proceed with the data collection.

The method of the invention further enables the measurement of the expression level of one or more of the proteins corresponding to the 41 genes according to the invention. Therefore, when step a) of the method of the invention comprises measuring the expression level of one or more of the proteins of the 41 genes according to the invention, the method of the invention comprises, upstream of step a), the step a′) of isolating said proteins from an isolated starting sample, said sample being a sample of ovarian tissue, blood and urine.

Step a′) is carried out according to known methods. The levels of one or more of the proteins isolated from the starting sample, e.g. an isolated biopsy of ovarian tissue, are then compared according to step c) of the method with the levels of the same proteins detected in isolated reference samples.

The method of the invention advantageously allowed to evaluate the average expression level of 41 genes in 42 samples of tumoral ovarian tissue and to compare it with the average expression level of the same genes in 3 normal ovarian tissues, said 3 normal ovarian tissues acting as reference samples, i.e. samples with which the expression level of the genes of interest of the isolated sample under investigation is corn pared.

Said 41 genes of which the expression value has been detected are ATP6V1B1, KRT18, KRT8, DSP, HMGA1, CLDN7, CLDN3, CLDN4, ELF3, ESRP1, MAL2, ST14, EPCAM, MYTH14, FAM83H, PRSS8, SPINT1, CELSR2, SORT1, GPR56/ADGRG1, WFDC2, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF, THSD4.

The level of the 41 genes determined in the 42 samples of tumoral tissue was compared with the expression level of the same genes in 3 healthy ovarian tissues, said 3 healthy ovarian tissues acting as reference samples according to step b) of the method of the invention.

According to step b) of the method of the invention, the expression level of at least one of the genes determined in step a) of the method is compared with the expression level of the same genes, determined in at least one reference sample. In other words, the expression level of each of the aforementioned 41 genes can be compared to the expression value of the same gene determined in a reference sample. Said reference sample may be an isolated sample selected from a sample of normal ovarian tissue or a sample of tumoral ovarian tissue. A sample of normal ovarian tissue, i.e. free of tumor condition, has physiological levels of gene expression. Said physiological expression levels can act as standard expression levels detected in healthy reference controls. Such standard expression levels may be, for example, a threshold value previously determined from the average of expression levels (obtained from multiple healthy samples of ovarian tissue) of a given gene or alternatively extracted from databases of various types.

In an embodiment, the at least one reference sample, against which the expression level of the genes of interest detected in the sample under investigation is compared, is a sample of tumoral ovarian tissue comprising gene expression levels different from those of a sample of normal ovarian tissue. In other words, the gene expression levels of the reference sample of tumoral ovarian tissue are not standard or physiological expression levels. Therefore, the method of the invention enables the comparison of the expression level of at least one of the genes selected from ATP6V1B1, KRT18, KRT8, DSP, HMGA1, CLDN7, CLDN3, CLDN4, ELF3, ESRP1, MAL2, ST14, EPCAM, MYH14, FAM83H, PRSS8, SPINT1, CELSR2, SORT1, GPR56/ADGRG1, WFDC2, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF, THSD4 by comparing it to the expression level of at least one of the same genes detected in a tumoral ovarian tissue or normal ovarian tissue, said tumoral ovarian tissue or normal ovarian tissue being reference samples.

In the present invention, the reference samples are samples of ovarian tissue (normal or tumoral) with which the expression level of at least one of the 41 genes detected in the isolated sample under investigation is compared.

According to an embodiment of the method of the invention, when the at least one reference sample according to step b) of the method is tumoral ovarian tissue, it is possible to assess a diagnosis of ovarian cancer when the isolated sample under investigation has expression levels of at least one of the 41 genes similar or equal to those detected for the reference sample.

According to another embodiment of the method of the invention, when the at least one reference sample according to step b) of the method is normal ovarian tissue, it is possible to assess a diagnosis of ovarian cancer when the isolated sample under investigation has expression levels of at least one of the 41 genes that are significantly different from those detected for the reference sample. According to another embodiment of the method of the invention, it is possible to assess a diagnosis of ovarian cancer when the expression level of at least one of the set forth 41 genes is found to be significantly higher in the isolated sample under investigation than in a normal reference ovarian tissue.

In fact, the method of the invention (carried out on 42 samples of tumoral ovarian tissue and 3 samples of normal ovarian tissue (reference samples according to step b) of the method)) has advantageously shown that the average amount of RNA of each of the 41 genes is 3 to 300 times higher in the isolated tumor samples than in the isolated samples of normal ovarian tissue (Example 1 and FIG. 1). In other words, the expression level of said 41 genes is always significantly higher than the expression level determined in isolated reference samples of normal ovarian tissue.

Alternatively, the expression levels of the reference sample can be extracted from databases or large-scale platforms of expression profiles, e.g. GTEx.

The validation of the 41 genes on 42 samples of tumoral ovarian tissue and 3 samples of normal ovarian tissue has shown that the detection of the expression level of said 41 genes according to the method of the invention is indicative of a diagnosis of ovarian cancer regardless of the set of samples analyzed and the RNA quantification technology.

Advantageously, the method of the invention also enables for a diagnosis of ovarian cancer to be made independently of the RNA quantification technology used.

Even more advantageously, the method of the invention allows to clearly discriminate the tumoral/normal origin of the isolated tissue analyzed. In fact, as shown in FIG. 2, the expression level of each of the 41 genes clearly identifies one condition versus another, e.g. tumoral condition versus normal condition. Since said 41 genes have an expression level 3 to 300 times higher than the expression level in healthy tissue, the expression level of even one of said 41 genes is capable of giving diagnostic indications.

Advantageously, the method of the invention enables for a diagnosis of ovarian cancer to be made independently of the RNA quantification technology used.

It has been observed that the amount of RNA produced by the 41 genes is low and homogeneous in the samples of normal ovarian tissue and high and extremely variable in the 42 tumoral tissues on which the method of the invention was tested.

Even more advantageously, the method of the invention carried out on 42 samples of tumoral ovarian tissue and 3 samples of isolated normal ovarian tissue allowed to identify 8 genes having the highest expression difference, said genes being EPCAM, ESRP1, GPR56/ADGRG1, MAL2, MYH14, PRSS8, ST14, WFDC2 (Example 2). In particular, said genes are 8 genes of the 41 genes of the method of the invention having a minimum expression level in the 42 samples of tumoral ovarian tissue at least 5 times higher than the maximum expression level in the 3 samples of normal ovarian tissue (FIG. 4).

According to a preferred embodiment, the method of the invention enables to diagnose tumoral ovarian tissues in a subject by determining, within one or more isolated samples, the levels of at least one gene selected from EPCAM, ESRP1, GPR56/ADGRG1, MAL2, MYH14, PRSS8, ST14, WFDC2, and/or of at least one protein encoded by said at least one gene.

According to a particularly preferred embodiment, the method of the invention enables to define the diagnosis of tumoral ovarian tissue in a subject by determining, within one or more isolated samples, the expression levels of at least one gene selected from EPCAM, ESRP1, GPR56/ADGRG1, MAL2, MYH14, PRSS8, ST14, WFDC2, and/or at least one protein encoded by said at least one gene, in combination with at least one of further genes selected from ATP6V1B1, KRT18, KRT8, DSP, HMGA1, CLDN7, CLDN3, CLDN4, ELF3, FAM83H, SPINT1, CELSR2, SORT1, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF, THSD4. In other words, according to the method of the invention, it is possible to detect the expression level of one or more of the 8 genes selected from EPCAM, ESRP1, GPR56/ADGRG1, MAL2, MYH14, PRSS8, ST14, WFDC2 in combination with one or more genes of the further 33 genes validated according to the method of the invention, said further 33 genes being genes that it is possible to determine in combination with the 8 preferred genes.

Once the expression level of at least one of the 8 genes, optionally in combination with one or more of 33 further genes, has been determined, said expression level is advantageously compared to the expression level of the same 8 genes, optionally the expression level of one or more of the further 33 genes, in at least one reference sample selected from samples of tumoral ovarian tissue and samples of isolated normal ovarian tissue. Alternatively, the expression level of at least one of the 8 genes, optionally in combination with one or more of 33 further genes, may be compared to the expression level of the same 8 genes, optionally the expression level of one or more of the further 33 genes, which is obtained from large-scale platforms of expression profiles.

The method of the invention according to this particularly preferred embodiment advantageously allows to have an accurate and certain diagnosis in a short time even on samples of tumoral ovarian tissue having extreme expression variability. According to a particularly preferred embodiment, the method of the invention may comprise detecting the expression level of the WFDC2 gene and/or of at least one protein encoded by said at least one gene, in combination with at least one of said further 33 genes. The method of the invention is a method having high specificity and sensitivity. In fact, the method of the invention exhibits 100% specificity and sensitivity when the expression level of one or more of the genes selected from EPCAM, ESRP1, GPR56/ADGRG1, MAL2, MYH14, PRSS8, ST14, WFDC2 is determined. Advantageously, a greater certainty of diagnosis of ovarian cancer is obtained when the determination of the expression level of one or more of said genes is combined with the determination of at least one of the aforementioned additional 33 genes. The combination of the expression level of at least one of the 8 genes with at least one of the 33 genes stated above is certainly advantageous when the expression level of one or more of the 8 detected genes has a value on the borderline between the physiological expression level, i.e. corresponding to that detected in healthy tissue, and the expression level of a tumoral tissue. In other words, when the expression level of one or more genes selected from EPCAM, ESRP1, GPR56/ADGRG1, MAL2, MYH14, PRSS8, ST14, WFDC2 is for example slightly above the physiological value detected for the same one or more genes, it is possible to determine the diagnosis of ovarian cancer by detecting the expression level of one or more of the aforementioned 33 genes so as to have another value on which to base a definite diagnosis.

Even more advantageously, the method according to the invention can also be fine-tuned to evaluate the outcomes of a surgical treatment to which a subject, who has already been diagnosed with ovarian cancer, may possibly be subjected. In fact, in order to ascertain that the tumoral mass has been completely removed, it is possible to perform the method of the invention on an isolated biopsy sample of the ovarian tissue after the surgery. In other words, after the surgery of resection or removal of the tumor, it will be possible to evaluate if there are still tumoral cells not removed by evaluating, on samples isolated from the ovarian tissue or from the surrounding tissues, the expression level of at least one of the 41 genes, according to the method of the invention. Therefore, in this case the isolated sample on which the method of the invention will be performed may be a biopsy sample of ovarian tissue taken by surgical removal of the part affected by the tumor. Furthermore, the method according to the invention also enables to evaluate the outcomes of a pharmacological treatment to which a subject, who has already been diagnosed with ovarian cancer, may be subjected. Also in this case the isolated sample on which the method of the invention may be performed may be a biopsy sample of ovarian tissue. A further advantageous application of the method of the invention relates to the evaluation of the expression level of said one or more 41 genes years after the onset of the tumor, to monitor and evaluate cases of recurrence, which are very frequent in patients with ovarian cancer, especially considering that 35-40% of patients survive 5 years after the diagnosis of ovarian cancer.

Surprisingly, out of the 41 genes selected by preliminary analysis (meta-analysis public platforms containing the expression profiles it was found that 8 genes (EPCAM, ESRP1, GPR56/ADGRG1, MAL2, MYH14, PRSS8, ST14, WFDC2) have prognostic value and that, therefore, can also identify the course of the disease, said disease being ovarian cancer. Therefore, the 8 genes selected from EPCAM, ESRP1, GPR56/ADGRG1, MAL2, MYH14, PRSS8, STI4, WFDC2, preferably WFDC2, as well as the proteins encoded by them, are significant in determining a prognosis in the presence of tumoral ovarian tissue. Therefore, said 8 genes are also useful in assessing the type of therapeutic treatment to which the patient with ovarian cancer should be subjected. In fact, on the basis of the prognosis formulated thanks to the determination of the expression levels of at least one of the 8 genes selected from EPCAM, ESRP1, GPR56/ADGRG1, MAL2, MYH14, PRSS8, ST14, WFDC2, and/or at least one protein encoded by said genes and the subsequent comparison of their expression level with the expression level of the same genes in reference samples selected from tumoral ovarian tissue and normal ovarian tissue, it is possible to identify the most appropriate therapy for the subject affected by ovarian cancer.

An object of the invention is also a method for defining the prognosis of a subject suffering from ovarian cancer comprising the steps of:
a) determining, in one or more isolated samples, the expression levels of at least one gene selected from EPCAM, ESRP1, GPR56/ADGRG1, MAL2, MYH14, PRSS8, ST14, WFDC2, preferably WFDC2, and/or of at least one protein encoded by said at least one gene; and
b) obtaining the expression levels of the same genes and/or of the same proteins in one or more reference samples
c) comparing the expression levels determined in step a), with the expression levels determined in step b).

In an alternative embodiment, the level of at least one of the 8 genes according to step a) of the method for defining the prognosis of a subject suffering from ovarian cancer can be determined in combination with at least one of the further 33 genes out of the 41 identified by preliminary analysis and validated according to the method of the invention, said 33 further genes being ATP6V1B1, KRT18, KRT8, DSP, HMGA1, CLDN7, CLDN3, CLDN4, ELF3, FAM83H, SPINT1, CELSR2, SORT1, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF, THSD4.

Further object of the present invention is a kit for the diagnosis of ovarian cancer, comprising one or more nucleic acid probes adapted to measure the expression levels of at least one gene selected from ATP6V1B1, KRT18, KRT8, DSP, HMGA1, CLDN7, CLDN3, CLDN4, ELF3, ESRP1, MAL2, ST14, EPCAM, MYH14, FAM83H, PRSS8, SPINT1, CELSR2, SORT1, GPR56/ADGRG1, WFDC2, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF, THSD4, at least one sample of reference nucleic acid, at least one buffer solution, preferably a hybridization buffer solution, and other media. According to a particularly preferred embodiment, said one or more nucleic acid probes are reporter probes and anchor probes according to the Nanostring nCounter DX Analysis System technology and said reference nucleic acid is a nucleic acid having gene expression levels of a tumoral ovarian tissue or a normal ovarian tissue.

According to said embodiment, the kit may have pairs of reporter probes and anchor probes for one or more of the 41 genes of the method of the invention or may have pairs of reporter probes and anchor probes for all 41 genes of the method of the invention, which are capable of hybridizing to the mRNA of one of the genes. According to a particularly preferred embodiment, the kit of the invention has pairs of reporter probes and anchor probes for at least one of 8 genes selected from EPCAM, ESRP1, GPR56/ADGRG1, MAL2, MYH14, PRSS8, ST14, WFDC2, preferably WFDC2, or alternatively has pairs of reporter probes and anchor probes for all 41 genes of the method of the invention that are capable of hybridizing to the mRNA of one of the genes. Being able to hybridize to the corresponding mRNA of said 41 genes, the reporter probes and anchor probes according to the kit of the invention have a sequence complementary to the sequence of the mRNAs of said 41 genes.

The kit of the invention may further comprise pairs of reporter probes and anchor probes for housekeeping genes, the expression level of which may be determined in order to normalize the expression level detected for the 41 genes of interest. For example, the kit of the invention may contain pairs of reporter probes and anchor probes for the housekeeping genes CLTC, GAPDH, GUSB, HPRT1, PGK1, TUBB. The kit of the invention may comprise pairs of reporter probes and anchor probes for other housekeeping genes, such as ACTB (beta-actin encoding gene).

The kit according to the invention comprises a sample of reference nucleic acid. Said sample of reference nucleic acid may be a sample of nucleic acid, e.g., RNA, comprising the expression levels of an ovarian cancer or a normal ovarian tissue. In other words, in an embodiment said sample of reference nucleic acid is a sample of isolated nucleic acid from a sample of isolated ovarian cancer. In another embodiment, the sample of reference nucleic acid is a sample of isolated nucleic acid from a sample of isolated normal ovarian tissue.

When the sample of reference nucleic acid in the kit of the invention is a sample of nucleic acid having gene expression levels of a tumoral ovarian tissue, the method of the invention allows a diagnosis of ovarian cancer to be made based on the similarity of the gene expression levels in the isolated sample under investigation to the expression levels of the sample of reference nucleic acid (from tumoral ovarian tissue). When the sample of reference nucleic acid in the kit of the invention is a sample of nucleic acid having gene expression levels of a normal ovarian tissue, the method of the invention enables a diagnosis of ovarian cancer to be made based on the difference in the gene expression levels in the isolated sample under investigation to the expression levels of the sample of reference nucleic acid (from normal ovarian tissue). The kit according to the invention may comprise other means such as, for example, tips, plates, test tubes, DEPC water. In a particularly preferred embodiment, the kit further includes a cartridge according to Nanostring technology and cartridge cover films.

Advantageously, the kit of the invention enabled the determination of the expression level of 41 genes selected on the basis of meta-analyses on public platforms of gene expression profiles and their comparison with the expression level of the same genes in non-tumoral samples according to the method of the invention. Furthermore, the kit of the invention enabled to identify 8 genes (out of 41 selected based on meta-analysis) having specificity and sensitivity of 100% in the set of samples analyzed. Therefore, the kit of the invention enables to implement a method for performing an in vitro diagnosis of ovarian cancer having specificity and sensitivity both equal to 100%.

The kit also enables the implementation of a method for the in vitro detection of the prognosis of a subject suffering from ovarian cancer having 100% sensitivity and specificity when the expression level of at least one of a gene selected from EPCAM, ESRP1, GPR56/ADGRG1, MAL2, MYH14, PRSS8, ST14, WFDC2, preferably WFDC2, is detected.

Advantageously, the Nanostring nCounter Analysis system also enables the quantitative determination of proteins present in a sample.

In fact, the molecular barcodes can also be used to quantify protein expression levels and are critical in the “protein assay”. The “protein assay” includes a mixture of antibodies in multiplex specific for the proteins of interest that have been barcoded with synthetic oligonucleotides (DNA). Each oligonucleotide is recognized by a unique Reporter probe that contains a fluorescent barcode. These Reporter probes are detected and quantified with an nCounter Analysis System that provides a direct digital readout of protein expression. After the sample preparation and probe hybridization, the samples are loaded onto the nCounter system for the assay execution and data collection. At this point, the barcodes are counted for the quantification of each protein of interest.

### BRIEF DESCRIPTION OF THE FIGURES

FIG. 1 depicts the amount of RNA of 41 genes in samples of normal (healthy) ovarian tissue and in samples of tumoral ovarian tissue.

FIG. 2 depicts the analysis of the main components of 41 differentially expressed genes in samples of normal ovarian tissue and in samples of tumoral ovarian tissue.

FIG. 3 depicts the heat map of 41 differentially expressed genes in 42 samples of tumoral ovarian tissue and in 3 samples of normal ovarian tissue.

FIG. 4 depicts the heat map of 8 differentially expressed genes in 42 samples of tumoral ovarian tissue and in 3 samples of normal ovarian tissue,

### DESCRIPTION OF THE FIGURES

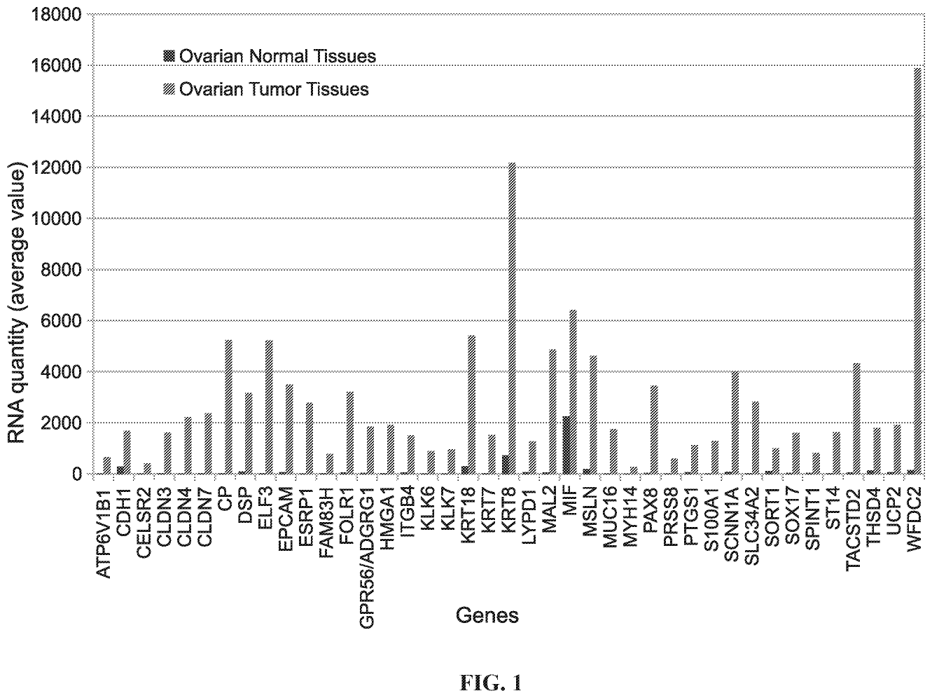

FIG. 1 depicts the amount of RNA of 41 genes in samples of normal ovarian tissue and in samples of tumoral ovarian tissue, expressed by the average value. On the ordinates the average value of RNA detected for each of the 41 genes depicted on the abscissa is represented. For each gene on the abscissa, the column in blue (first column for each gene) depicts the average value of mRNA corresponding to the gene in the normal ovarian tissue, whereas the column in orange (second column for each gene) depicts the average value of mRNA corresponding to the gene in the tumoral ovarian tissue.

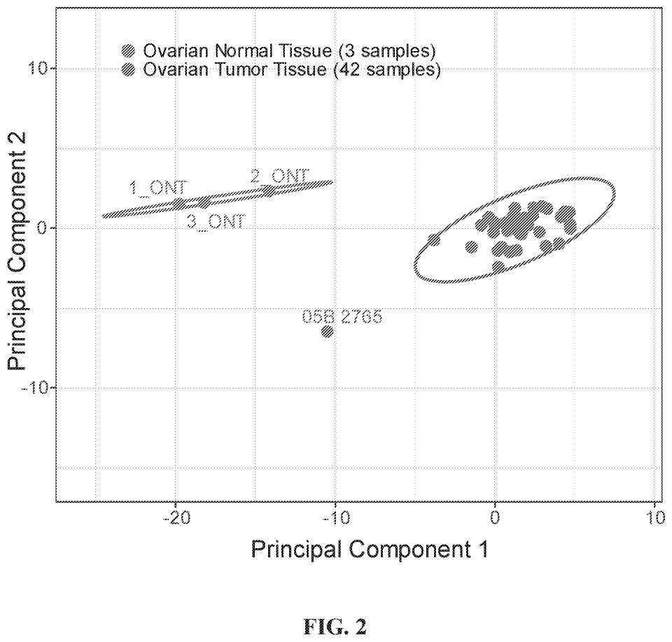

FIG. 2 depicts the main component analysis of 41 differentially expressed genes in samples of normal (healthy) ovarian tissue and in samples of tumoral ovarian tissue, clearly highlighting that said 41 differentially expressed genes selected for the method of the invention allow discriminating the tumoral/normal origin of the analyzed tissue. On the top left the normal ovarian tissues 1_ONT, 2_ONT, 3_ONT (in green) are depicted, whereas on the right of the graph the samples of tumoral ovarian tissue (in orange) are depicted.

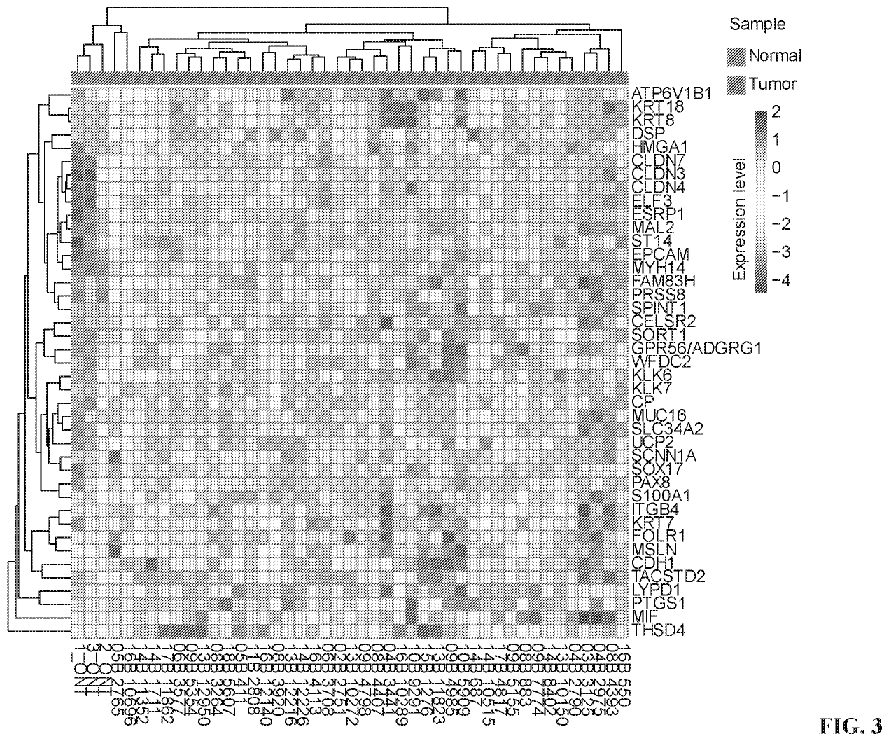

FIG. 3 depicts the heat map of 41 differentially expressed genes in 42 samples of tumoral ovarian tissue and 3 samples of normal ovarian tissue. On the abscissae the 3 samples of normal ovarian tissue (1_ONT, 2_ONT, 3_ONT, on the left) and the 42 samples of tumoral ovarian tissue (05B2765 to 16B550) are placed according to example 1 whereas on the ordinates the 41 genes are listed. The amount of RNA of the 41 analyzed genes is extremely low in the 3 samples of normal ovarian tissue 1_ONT, 2_ONT, 3_ONT (on the left of the graph) with which an expression level from −4 to −1 is associated, whereas it is variable in the 42 ovarian tissues (from 05B2765 to 16B550) with an expression value ranging from 2 to −1 and never below −2. The expression level from −4 to −1 of the samples of normal ovarian tissue is shown in FIG. 3 with a blue color scale (from light blue corresponding to a value of −2 to deep blue corresponding to a value of −4), whereas the expression level from samples of tumoral ovarian tissue is shown in FIG. 3 with a red color scale (from light orange corresponding to a value of 0 to deep red corresponding to a value of 2). The expression value corresponding to −1 in FIG. 3 is depicted by white color.

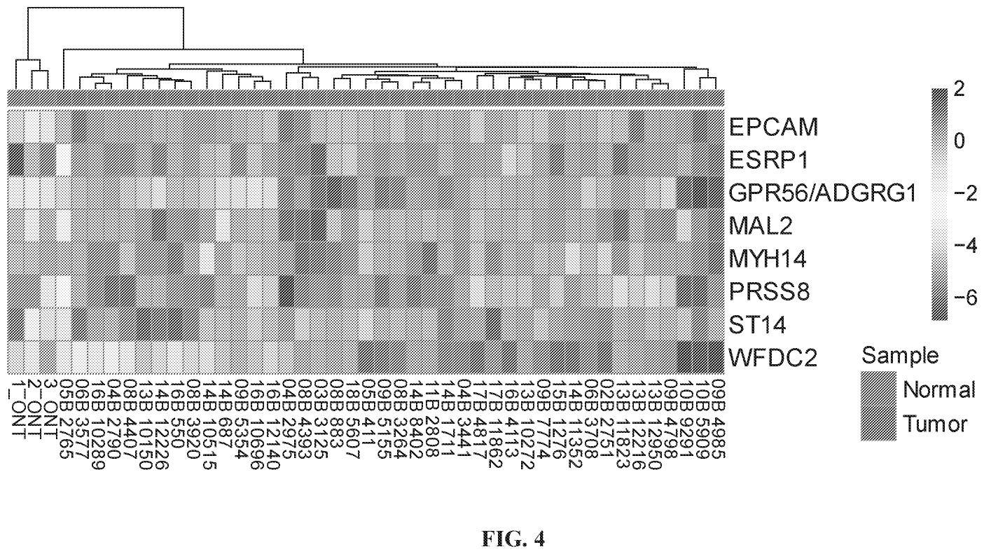

FIG. 4 depicts the heat map of 8 differentially expressed genes in 42 samples of tumoral ovarian tissue and in 3 samples of normal ovarian tissue. Said 8 genes are genes whose minimum expression level in the 42 tumor samples was at least 5 times higher than the maximum expression level in the 3 samples of normal ovarian tissue, e.g., genes over-expressed in the tumoral ovarian tissue. On the abscissae the 3 samples of normal ovarian tissue (1_ONT, 2_ONT, 3_ONT, on the left) and the 42 samples of tumoral ovarian tissue (05B2765 to 16B550) are placed, whereas on the ordinates the genes are listed. The amount of RNA of the 41 analyzed genes is extremely low in the 3 samples of normal ovarian tissue (1_ONT, 2_ONT, 3_ONT, on the left) with which an expression level from −6 to −2 is associated, whereas it is considerably higher in the 42 ovarian tissues (from 05B2765 to 16B550) with an expression value ranging from 0 to 2.

The expression level from −6 to −2 of the 8 genes in the samples of normal ovarian tissue is shown in FIG. 4 with a blue color scale (from light blue corresponding to a value of −2 to deep blue corresponding to a value of −4), whereas the expression level of the 8 genes in the samples of tumoral ovarian tissue is shown in FIG. 4 with a red color scale (from light orange corresponding to a value equal to 0 to deep red corresponding to a value of 2). The expression value corresponding to −1 in FIG. 4 is depicted by white color.

### Experimental Section

In the present experimental section will be reported, in the order in which they were performed, the tests and analyses that allowed the identification of genes differentially expressed in the healthy ovarian tissues and isolated ovarian tumors.

### Example 1

Comparative Analysis of the Expression of 41 Genes (Selected by Preliminary Analysis) in Samples of Tumoral Ovarian Tissue Versus Samples of Normal Ovarian Tissue

For the present comparative analysis, biopsy samples were made available: 42 FFPE (formalin fixed paraffin-embedded) samples of tumoral ovarian tissue and 3 FFPE (formalins fixed paraffin-embedded) samples of healthy ovarian tissue. The total RNA was extracted from 10 μm sections of said biopsy samples by using High Pure FFPET RNA Isolation Kit (Roche). RNA was quantified with BioPhotometer® 030 (Eppendorf) and 100 ng of total RNA was analyzed with Nanostring nCounter Analysis System technology. This system uses a new technology based on colored barcodes which allow direct multiplex measurements of gene expression, which are characterized by high levels of precision and sensitivity (<1 copy/cell).

The nCounter CodeSet used contained a pair of probes (reporter probe and anchor probe) specific for each of the 41 genes identified by meta-analysis on the TCGA and GTEx gene expression platforms and for 6 housekeeping genes (CLTC, GAPDH, GUSB, HPRT1, PGK1, TUBB) inserted for the normalization of the expression values.

The Nanostring nCounter analysis was performed following the instructions of the supplier (www.nanostring.com). In particular, 100 ng of total RNA was hybridized in solution at 65° C. for 16 hours with a specific pair of probes in the CodeSet, each of 50 bases, for each of the mRNAs in the sample.

The probe pair for each mRNA was constituted by a reporter probe having the barcode (e.g., color code) and an anchor probe to immobilize the probe+mRNA complex on the nCounter cartridge for data collection.

The reporter probe used in said Nanostring technology is constituted by a sequence of 50 base pairs complementary to the mRNA of interest and a DNA sequence that hybridizes the 6 tagged RNA segments with one of the four fluorescent markers: red, yellow, blue or green, whereas the anchor probe is constituted by a sequence of about 50 bases complementary to the target mRNA and ends with a biotin molecule used for the immobilization on the streptavidin-coated nCounter cartridge. After the probe hybridization was achieved, the reporter probes and excess anchor probes were removed by an automated procedure using magnetic beads and then the probe/mRNA complexes were retained on the cartridge surface by strepta.vidin-biotin binding. Finally, the probe/mRNA complexes were aligned and immobilized on the cartridge. The cartridge was placed in the nCounter Digital Analyzer for the data collection. The colored barcodes on the surface of the cartridge were read and tabulated for each mRNA molecule. Each target molecule of interest is identified by the “color code” generated by the six fluorescent dots present on the respective associated reporter probe. The measured value of each mRNA was normalized for the measured value for the housekeeping genes.

The method of the invention performed on 42 samples of tumoral ovarian tissue and 3 samples of normal ovarian tissue allowed to evaluate the average amount of RNA of each of the 41 genes is 3 to 300 times higher in the tumoral samples than in the healthy tissue samples (not affected by cancer) (FIG. 1).

It was also observed that the amount of RNA produced by the 41 genes is extremely low and homogeneous in the 3 normal tissues, whereas it is more variable in the 42 tumoral tissues (FIG. 3).

### Example 2

Comparative Analysis of the Expression of 8 Genes Selected by Preliminary Analysis in Samples of Tumoral Ovarian Tissue Versus Samples of Normal Ovarian Tissue

The method of the invention was tested on 42 samples of tumoral ovarian tissue (formalin-fixed paraffin-embedded) and 3 samples of normal ovarian tissue (formalin-fixed paraffin-embedded) by using Nanostring technology according to Example 1. The expression value of the 41 genes identified by preliminary analysis on public gene expression platforms was identified and normalized for the expression value of 6 housekeeping genes (CLTC, GAPDH, GUSB, HPRT1, PGK1, TUBB).

From the present study comparing the expression profile of the 42 samples of tumoral ovarian tissue and the 3 healthy tissue samples, 8 genes were selected having an expression level 5 times higher than the maximum expression level in the 3 samples of normal ovarian tissue.

Therefore, the method of the invention allowed to identify 8 genes which individually exhibit specificity and sensitivity of 100% in the set of samples analyzed.

Advantageously, these 8 genes enable to obtain an accurate diagnosis even on samples of tumoral ovarian tissue having an extreme variability of expression as it has been observed that their expression level is always higher than the expression level of the corresponding gene in samples of healthy ovarian tissue (FIG. 4).

1. A method for carrying out an in vitro diagnosis of ovarian cancer in a subject, comprising the steps of:
b) determining, in one or more isolated samples, the expression levels of at least one gene and/or of at least one protein encoded by said at least one gene;
c) obtaining the expression levels of the same genes and/or of the same proteins in one or more isolated reference samples; and
d) comparing the expression levels determined in step a), with the expression levels determined in step b),
wherein said at least one gene is selected from ATP6V1B1, KRT18, KRT8, DSP, HMGA1, CLDN7, CLDN3, CLDN4, ELF3, ESRP1, MAL2, ST14, EPCAM, MYH14, FAM83H, PRSS8, SPINT1, CELSR2, SORT1, GPR56/ADGRG1, WFDC2, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF and THSD4.

2. The method according to claim 1, wherein said at least one gene is selected from EPCAM, ESRP1, GPR56/ADGRG1, MAL2, MYH14, PRSS8, ST14, and WFDC2.

3. The method according to claim 1, wherein said at least one gene is selected from EPCAM, ESRP1, GPR56/ADGRG1, MAL2, MYH14, PRSS8, ST14, and WFDC2, and said further genes are selected from ATP6V1B1, KRT18, KRT8, DSP, HMGA1, CLDN7, CLDN3, CLDN4, ELF3, FAM83H, SPINT1, CELSR2, SORT1, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF and THSD4.

4. The method for defining in vitro the prognosis of a subject suffering from tumoral ovarian tissue, comprising the steps of:
a) determining, in one or more isolated samples, the expression levels of at least one gene selected from EPCAM, ESRP1, GPR56/ADGRG1, MAL2, MYH14, PRSS8, ST14, WFDC2, and/or of at least one protein encoded by said at least one gene;
b) obtaining the expression levels of the same genes and/or of the same proteins in one or more isolated reference samples;
c) comparing the expression levels determined in step a), with the expression levels determined in step b).

5. The method according to claim 4, wherein said one or more isolated samples are selected from ovarian tissue sample, blood and urine and said at least one reference sample according to step b) of the method is an isolated sample selected from a sample of tumoral ovarian tissue and a sample of healthy ovarian tissue.

6. The method according to claim 4, wherein said expression levels are expression levels of the RNA of said at least one gene.

7. The method according to claim 4, wherein step a) is carried out on an analysis platform of the gene expression level.

8. A kit for the in vitro diagnosis of ovarian tumor according to the method of claim 1, comprising one or more nucleic acid probes adapted to measure the expression levels of at least one gene selected from ATP6V1B1, KRT18, KRT8, DSP, HMGA1, CLDN7, CLDN3, CLDN4, ELF3, ESRP1, MAL2, ST14, EPCAM, MYH14, FAM83H, PRSS8, SPINT1, CELSR2, SORT1, GPR56/ADGRG1, WFDC2, KLK6, KLK7, CP, MUC16, SLC34A2, UCP2, SCNN1A, SOX17, PAX8, S100A1, ITGB4, KRT7, FOLR1, MSLN, CDH1, TACSTD2, LYPD1, PTGS1, MIF and THSD4, at least one sample of nucleic acid of reference and at least one buffer solution, and other media.

9. The kit to assess in vitro the prognosis of ovarian tumor according to the method of claim 4, comprising one or more nucleic acid probes adapted to measure the expression levels of at least one gene selected from EPCAM, ESRP1, GPR56/ADGRG1, MAL2, MYH14, PRSS8, ST14 and WFDC2 at least one sample of nucleic acid of reference and at least one buffer solution.

10. The kit according to claim 8, wherein said one or more nucleic acid probes are reporter probes and anchor probes according to the Nanostring nCounter DX Analysis System technology and said nucleic acid of reference is a nucleic acid having gene expression levels of an ovarian carcinoma or a healthy ovarian tissue.

11. The method according to claim 1, wherein said at least one gene is WFDC2.

12. The method according to claim 4, wherein said at least one gene is WFDC2.

13. The method according to claim 7, wherein step a) is carried out on an nCounter DX Analysis System platform.

14. The kit for the in vitro diagnosis of ovarian tumor according to of claim 8, wherein the at least one buffer solution is a hybridization buffer solution.

15. The kit according to claim 9, wherein said one or more nucleic acid probes are reporter probes and anchor probes according to the Nanostring nCounter DX Analysis System technology and said nucleic acid of reference is a nucleic acid having gene expression levels of an ovarian carcinoma or a healthy ovarian tissue.
